(19) United States
(12) Patent Application Publication
(10) Pub. No.: US 2015/0099802 A1
(43) Pub. Date: Apr. 9, 2015

(54) SELECTIVE TUMOR TREATMENT

(71) Applicant: SUCAMPO AG, Zug (CH)

(72) Inventors: Ryuji UENO, Easton, MD (US); Sachiko KUNO, Easton, MD (US)

(73) Assignee: SUCAMPO AG, Zug (CH)

(21) Appl. No.: 14/503,922

(22) Filed: Oct. 1, 2014

Related U.S. Application Data
(60) Provisional application No. 61/886,307, filed on Oct. 3, 2013.

(51) Int. Cl. C07D 311/94; C12Q 1/68; G01N 33/573; C12Q 1/32

(52) U.S. Cl. CPC C07D 311/94; C12Q 1/32; C12Q 1/6886; G01N 33/573; G01N 2333/904; C12Q 2600/158; C12Q 2600/106; G01N 2800/52
USPC 514/456; 435/6.12; 435/7.4; 435/375

(57) ABSTRACT

A method for treating a tumor with a decreased level of the 15-hydroxy-prostaglandin dehydrogenase (15-PGDH) in a mammalian subject, which comprises administering to the subject in need thereof an effective amount of a fatty acid derivative, a method for suppressing the growth of a tumor cell with a decreased level of the 15-hydroxy-prostaglandin dehydrogenase (15-PGDH), which comprises contacting said tumor cell with an effective amount of a fatty acid derivative, and a method for identifying a subject who would be responsive to a fatty acid derivative, comprising, (i) obtaining a biological sample from said subject; and (ii) measuring 15-hydroxyprostaglandin dehydrogenase (15-PGDH) level are provided.

### CROSS REFERENCE TO RELATED APPLICATIONS

This application claims the benefit of U.S. Provisional Patent Application No. 61/886,307 filed Oct. 3, 2013, the entire contents of which are incorporated by reference herein.

### TECHNICAL FIELD

The present invention relates to a method for treating a tumor with a decreased level of the 15-hydroxy-prostaglandin dehydrogenase (15-PGDH) in a mammalian subject with an effective amount of a fatty acid derivative.

### BACKGROUND

15-hydroxy-prostaglandin dehydrogenase (15-PGDH) is an enzyme that converts PGE₂ into 15-keto-prostaglandin by working on 15(S)-hydroxyl groups, eventually leading to inactivation of PGE₂. Recent studies have demonstrated that decreased 15-PGDH has a profound relationship with carcinogenesis and cancer progression. Decreased 15-PGDH expression was observed in patients with colorectal cancer, breast cancer, prostate cancer, lung cancer, thyroid cancer, gastric cancer and other cancers. Kang et al. reported that 15-PGDH was down-regulated in 55.8% of the colorectal cancer patients (Clin Cancer Res 2009; 15(14), 2009 and J Korean Soc Coloproctol 2012; 28(5): 253-258).

Fatty acid derivatives are members of class of organic carboxylic acids, which are contained in tissues or organs of human or other mammals, and exhibit a wide range of physiological activity. Some fatty acid derivatives found in nature generally have a prostanoic acid skeleton as shown in the formula (A):

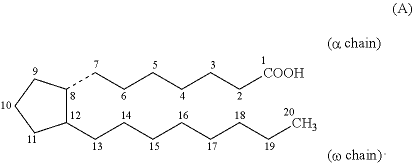

On the other hand, some of synthetic prostaglandin (PG) analogues have modified skeletons. The primary PGs are classified into PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-0H

Subscript 2: 5,6- and 13,14-diunsaturated-15-0H

Subscript 3: 5,6-, 13,14-, and 17,18-triunsaturated-15-OH.

Further, the PGFs are classified, according to the configuration of the hydroxyl group at the 9-position, into α type (the hydroxyl group is of an α-configuration) and β type (the hydroxyl group is of a β-configuration).

PGs are known to have various pharmacological and physiological activities, for example, vasodilatation, inducing of inflammation, platelet aggregation, stimulating uterine muscle, stimulating intestinal muscle, anti-ulcer effect and the like.

Prostones, having an oxo group at position 15 of prostanoic acid skeleton (15-keto type) and having a single bond between positions 13 and 14 and an oxo group at position 15 (13,14-dihydro-15-keto type), are fatty acid derivatives known as substances naturally produced by enzymatic actions during metabolism of the primary PGs and have some therapeutic effect.

US patent publication No. 2006/0281818 to Ueno et al. discloses specific prostaglandin compounds are useful for treating mucosal disorders including cancer.

However it is not known how specific fatty acid derivatives act on the specific type of tumors.

### DISCLOSURE OF THE INVENTION

The present invention relates to a method for treating a tumor with a decreased level of the 15-hydroxy-prostaglandin dehydrogenase in a mammalian subject, which comprises administering to the subject in need thereof an effective amount of a fatty acid derivative represented by the formula (I):

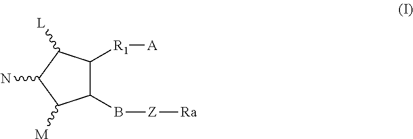

wherein L, M and N are hydrogen, hydroxy, halogen, lower alkyl, hydroxy(lower)alkyl, lower alkanoyloxy or oxo, wherein the five-membered ring may have at least one double bond;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C≡CH₂— or —CH₂—C≡C—;

Z is

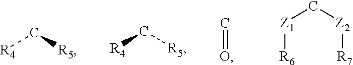

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₂ and Z₂ are oxygen, nitrogen or sulfur; R₆ and R₇ are optionally substituted lower alkyl, which is optionally linked together to form lower alkylene;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

The present invention also relates to a method for suppressing the growth of a tumor cell with a decreased level of the 15-hydroxy-prostaglandin dehydrogenase (15-PGDH), which comprises contacting said tumor cell with an effective amount of a fatty acid derivative disclosed herein.

The present invention further relates to a method for identifying a subject who would be responsive to a fatty acid derivative, comprising,

(i) obtaining a biological sample from the subject; and
(ii) measuring 15-hydroxyprostaglandin dehydrogenase (15-PGDH) level.

### BRIEF DESCRIPTION OF DRAWINGS

FIG. 1A shows effect of lubiprostone on growth of Non-transfected colon adenocarcinoma LS174T cells. Data were plotted as mean±SEM, n=3 for each experimental condition.

FIG. 1B shows effect of lubiprostone on growth of hPGDH-transfected colon adenocarcinoma LS174T cells. Data were plotted as mean±SEM, n=3 for each experimental condition.

FIG. 1C shows effect of lubiprostone on growth of Mock-transfected colon adenocarcinoma LS174T cells. Data were plotted as mean±SEM, n=3 for each experimental condition.

### DETAILED DESCRIPTION OF THE INVENTION

The nomenclature of the fatty acid derivative used herein is based on the numbering system of the prostanoic acid represented in the above formula (A).

The formula (A) shows a basic skeleton of the C-20 fatty acid derivative, but the present invention is not limited to those having the same number of carbon atoms. In the formula (A), the numbering of the carbon atoms which constitute the basic skeleton of the fatty acid derivatives starts at the carboxylic acid (numbered 1), and carbon atoms in the α-chain are numbered 2 to 7 towards the five-membered ring, those in the ring are 8 to 12, and those in the co-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of carboxy group (C-1). Similarly, when the number of carbon atoms is decreased in the co-chain, the number is deleted in the order starting from position 20; and when the number of carbon atoms is increased in the ω-chain, the carbon atoms at the position or later are named as a substituent at position 20. Stereochemistry of the compounds is the same as that of the above formula (A) unless otherwise specified.

In general, each of PGD, PGE and PGF represents a fatty acid derivative having hydroxy groups at positions 9 and/or 11, but in the present specification they also include those having substituents other than the hydroxy groups at positions 9 and/or 11. Such compounds are referred to as 9-deoxy-9-substituted-fatty acid derivatives or 11-deoxy-11-substituted-fatty acid derivatives. A fatty acid derivative having hydrogen in place of the hydroxy group is simply named as 9- or 11-deoxy-fatty acid derivative.

As stated above, the nomenclature of a fatty acid derivative is based on the prostanoic acid skeleton. In the case the compound has similar partial structure as the primary PG, the abbreviation of “PG” may be used. Thus, a fatty acid derivative whose α-chain is extended by two carbon atoms, that is, having 9 carbon atoms in the α-chain is named as 2-decarboxy-2-(2-carboxyethyl)-PG compound. Similarly, a fatty acid derivative having 11 carbon atoms in the α-chain is named as 2-decarboxy-2-(4-carboxybutyl)-PG compound. Further, a fatty acid derivative whose co-chain is extended by two carbon atoms, that is, having 10 carbon atoms in the co-chain is named as 20-ethyl-PG compound. These compounds, however, may also be named according to the IUPAC nomenclatures.

Examples of the analogues including substitution compounds or derivatives of the above described fatty acid derivative include a fatty acid derivative whose carboxy group at the end of the alpha chain is esterified; a fatty acid derivative whose α chain is extended, a physiologically acceptable salt thereof, a fatty acid derivative having a double bond between positions 2 and 3 or a triple bond between positions 5 and 6; a fatty acid derivative having substituent(s) on carbon atom(s) at position(s) 3, 5, 6, 16, 17, 18, 19 and/or 20; and a fatty acid derivative having a lower alkyl or a hydroxy (lower) alkyl group at position 9 and/or 11 in place of the hydroxy group.

According to the present invention, preferred substituents on the carbon atom at position(s) 3, 17, 18 and/or 19 include alkyl having 1-4 carbon atoms, especially methyl and ethyl. Preferred substituents on the carbon atom at position 16 include lower alkyls such as methyl and ethyl, hydroxy, halogen atom such as chlorine and fluorine, and aryloxy such as trifluoromethylphenoxy. Preferred substituents on the carbon atom at position 17 include lower alkyl such as methyl and ethyl, hydroxy, halogen atom such as chlorine and fluorine, and aryloxy such as trifluoromethylphenoxy. Preferred substituents on the carbon atom at position 20 include saturated or unsaturated lower alkyl such as C₁₋₄ alkyl, lower alkoxy such as C₁₋₄ alkoxy, and lower alkoxy alkyl such as C₁₋₄ alkoxy-C₁₋₄ alkyl. Preferred substituents on the carbon atom at position 5 include halogen atoms such as chlorine and fluorine. Preferred substituents on the carbon atom at position 6 include an oxo group forming a carbonyl group. Stereochemistry of PGs having hydroxy, lower alkyl or hydroxy(lower)alkyl substituent on the carbon atom at positions 9 and 11 may be α, β or a mixture thereof.

Further, the above described analogues or derivatives may have a co chain shorter than that of the primary PGs and a substituent such as alkoxy, cycloalkyl, cycloalkyloxy, phenoxy and phenyl at the end of the truncated ω-chain.

A fatty acid derivative used in the present invention is represented by the formula (I):

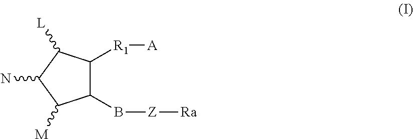

wherein L, M and N are hydrogen, hydroxy, halogen, lower alkyl, hydroxy(lower)alkyl, lower alkanoyloxy or oxo, wherein the five-membered ring may have at least one double bond;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

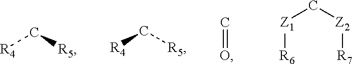

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

A preferred compound used in the present invention is represented by the formula (II):

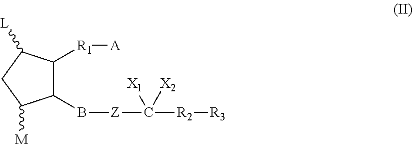

wherein L and M are hydrogen atom, hydroxy, halogen, lower alkyl, hydroxy(lower)alkyl, lower alkanoyloxy or oxo, wherein the five-membered ring may have one or more double bonds;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

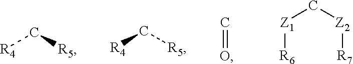

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;

X₁ and X₂ are hydrogen, lower alkyl, or halogen;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur;

R₂ is a single bond or lower alkylene; and

R₃ is lower alkyl, lower alkoxy, lower alkanoyloxy, cyclo(lower)alkyl, cyclo(lower)alkyloxy, aryl, aryloxy, heterocyclic group or heterocyclic-oxy group, and at least one of carbon atom in the aliphatic hydrocarbon is optionally substituted by oxygen, nitrogen or sulfur.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, isopropylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydrogen, hydroxy and oxo, and especially, L and M are both hydroxy, or L is oxo and M is hydrogen or hydroxy.

Preferred example of A is —COOH, its pharmaceutically acceptable salt, ester or amide thereof.

Preferred example of X₁ and X₂ are both being hydrogens or halogen atoms, and in case of halogen atoms, more preferably, fluorine atoms, so called 16,16-difluoro type.

Preferred R₁ is a hydrocarbon residue containing 1-10 carbon atoms, preferably 6-10 carbon atoms. Further, at least one carbon atom in the aliphatic hydrocarbon is optionally substituted by oxygen, nitrogen or sulfur. Examples of R₁ include, for example, the following groups:
—CH₂—CH₂—CH₂—CH₂—CH₂—CH₂—,
—CH₂—CH═CH—CH₂—CH₂—CH₂—,
—CH₂—CH₂—CH₂—CH₂—CH═CH—,
—CH₂—C═C—CH₂—CH₂—CH₂—,
—CH₂—CH₂—CH₂—CH₂—O—CH₂—,
—CH₂—CH═CH—CH₂—O—CH₂—,
—CH₂—C═C—CH₂—O—CH₂—,
—CH₂—CH₂—CH₂—CH₂—CH₂—CH₂—CH₂—,
—CH₂—CH═CH—CH₂—CH₂—CH₂—CH₂—,
—CH₂—CH₂—CH₂—CH₂—CH₂—CH═CH—,
—CH₂—C═C—CH₂—CH₂—CH₂—CH₂—,
—CH₂—CH₂—CH₂—CH₂—CH₂—CH(CH₃)—CH₂—,
—CH₂—CH₂—CH₂—CH₂—CH(CH₃)—CH₂—,
—CH₂—CH₂—CH₂—CH₂—CH₂—CH₂—CH₂—CH₂—,
—CH₂—CH═CH—CH₂—CH₂—CH₂—CH₂—CH₂—,
—CH₂—CH₂—CH₂—CH₂—CH₂—CH₂—CH═CH—,
—CH₂—C≡C—CH₂—CH₂—CH₂—CH₂—CH₂—, and
—CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains having one carbon atom. Further, at least one carbon atom in the aliphatic hydrocarbon is optionally substituted by oxygen, nitrogen or sulfur.

In embodiments of the present invention, representative compounds of the formula (I) or (II) include compounds of the formula (I) wherein Ra is substituted by halogen and/or Z is C═O;

compounds of the formula (II) wherein one of X₁ and X₂ is substituted by halogen and/or Z is C═O;
compounds of the formula (II) wherein L is ═O or —OH, M is H or OH, A is COOH or a functional derivative thereof, B is —CH₂—CH₂—, Z is C═O, X₁ is halogen (e.g. X₁ is Cl, Br, I or F) or hydrogen, X₂ is halogen (e.g. X₂ is Cl, Br, I or F) or hydrogen, R₁ is a saturated or unsaturated bivalent straight C₆ aliphatic hydrocarbon residue, R₂ is a single bond, and R₃ is straight or branched lower alkyl (e.g. C₄₋₆ alkyl) optionally substituted by oxygen, nitrogen or sulfur;
compounds of the formula (II) wherein L is ═O, M is OH, A is COOH or a functional derivative thereof, B is —CH₂—CH₂—, Z is C═O, X₁ is halogen (e.g. X₁ is Cl, Br, I or F) or hydrogen, X₂ is halogen (e.g. X₂ is Cl, Br, I or F) or hydrogen, R₁ is a saturated or unsaturated bivalent straight C₆ aliphatic hydrocarbon residue, R₂ is a single bond, and R₃ is straight or branched lower alkyl optionally substituted by oxygen, nitrogen or sulfur;
compounds of the formula (II) wherein L is ═O, M is OH, A is COOH or a functional derivative thereof, B is —CH₂—CH₂—, Z is C═O, X₁ and X₂ are halogen atoms (e.g. X₁ and X₂ are Cl, Br, I or F), R₁ is a saturated or unsaturated bivalent straight C₆ aliphatic hydrocarbon residue, R₂ is a single bond, and R₃ is straight or branched lower alkyl (e.g. C₄ alkyl or C₅ alkyl);
compounds of the formula (II) wherein L is ═O, M is OH, A is COOH or a functional derivative thereof, B is —CH₂—CH₂—, Z is C═O, X₁ and X₂ are fluorine atoms, R₁ is a saturated or unsaturated bivalent straight C₆ aliphatic hydrocarbon residue, R₂ is a single bond, and R₃ is straight or branched lower alkyl (e.g. C₄ alkyl or C₅ alkyl);
compounds of the formula (II) wherein L is ═O, M is H or OH, A is COOH or a functional derivative thereof, B is —CH₂—CH₂—, Z is C═O, X₁ and X₂ are halogen atoms (e.g. X₁ and X₂ are Cl, Br, I or F), R₁ is a saturated or unsaturated bivalent straight C₆ aliphatic hydrocarbon residue, R₂ is a single bond, and R₃ is —CH₂—CH₂—CH₂—CH₃ or —CH₂—CH(CH₃)—CH₂—CH₃.

In further embodiment, representative compounds used in the present invention include (−)-7-[(2R,4aR,5R,7aR)-2-(1,1-difluoropentyl)-2-hydroxy-6-oxooctahydrocyclopenta[b]pyran-5-yl]heptanoic acid (lubiprostone), (−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid (cobiprostone), (+)-isopropyl (Z)-7-[(1R,2R,3R,5S)-3,5-dihydroxy-2-(3-oxodecyl)cyclopentyl]hept-5-enoate (isopropyl unoprostone), (Z)-7-[(1R,2R,3R,5S)-3,5-dihydroxy-2-(3-oxodecyl)cyclopentyl]hept-5-enoic acid, (−)-7-[(1R,2R)-2-(4,4-difluoro-3-oxooctyl)-5-oxocyclopentyl]heptanoic acid, (E)-7-[(1R,2R)-2-(4,4-difluoro-3-oxooctyl)-5-oxocyclopentyl]hept-2-enoic acid, an isomer (including tautomeric isomer) thereof and a functional derivative thereof.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

In the present invention, the fatty acid derivative which is dihydro between 13 and 14, and keto(═O) at 15 position may be in the keto-hemiacetal equilibrium by formation of a hemiacetal between hydroxy at position 11 and keto at position 15.

For example, it has been revealed that when both of X₁ and X₂ are halogen atoms, especially, fluorine atoms, the compound contains a tautomeric isomer, bicyclic compound.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the present invention includes both isomers.

Further, the fatty acid derivatives used in the invention include the bicyclic compound and analogs or derivatives thereof.

The bicyclic compound is represented by the formula (III)

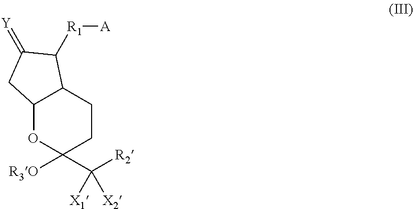

wherein, A is —CH₃, or —CH₂OH, —COCH₂OH, —COOH or a functional derivative thereof;

X₁′ and X₂′ are hydrogen, lower alkyl, or halogen;

Y is

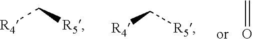

wherein R₄′ and R₅′ are hydrogen, hydroxy, halogen, lower alkyl, lower alkoxy or hydroxy(lower)alkyl, wherein R₄′ and R₅′ are not hydroxy and lower alkoxy at the same time.

R₁ is a saturated or unsaturated divalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

R₂′ is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

R₃′ is hydrogen, lower alkyl, cyclo(lower)alkyl, aryl or heterocyclic group.

Furthermore, while the compounds used in the invention may be represented by a formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324, 5,739,161, 6,242,485 and 8,202,909 (these cited references are herein incorporated by reference).

The mammalian subject may be any mammalian subject including a human. The compound may be applied systemically or topically. Usually, the compound may be administered by oral administration, intranasal administration, inhalational administration, intravenous injection (including infusion), subcutaneous injection, ocular topical administration, intra rectal administration, intra vaginal administration, transdermal administration and the like.

The dose may vary depending on the strain of the animal, age, body weight, symptom to be treated, desired therapeutic effect, administration route, term of treatment and the like. A satisfactory effect can be obtained by systemic administration 1-4 times per day or continuous administration at the amount of 0.00001-500 mg/kg per day, more preferably 0.0001-100 mg/kg.

The compound may preferably be formulated in a pharmaceutical composition suitable for administration in a conventional manner. The composition may be those suitable for oral administration, intranasal administration, ocular topical administration, inhalational administration, injection or perfusion as well as it may be an external agent, suppository or pessary.

The composition of the present invention may further contain physiologically acceptable additives. The additives may include the ingredients used with the present compounds such as excipient, diluent, filler, resolvent, lubricant, adjuvant, binder, disintegrator, coating agent, cupsulating agent, ointment base, suppository base, aerozoling agent, emulsifier, dispersing agent, suspending agent, thickener, tonicity agent, buffering agent, soothing agent, preservative, antioxidant, corrigent, flavor, colorant, a functional material such as cyclodextrin and biodegradable polymer, stabilizer. The additives are well known to the art and may be selected from those described in general reference books of pharmaceutics.

The amount of the above-defined compound in the composition of the invention may vary depending on the formulation of the composition, and may generally be 0.000001-10.0%, more preferably 0.00001-5.0%, most preferably 0.0001-1%.

Examples of solid compositions for oral administration include tablets, troches, sublingual tablets, capsules, pills, powders, granules and the like. The solid composition may be prepared by mixing one or more active ingredients with at least one inactive diluent. The composition may further contain additives other than the inactive diluents, for example, a lubricant, a disintegrator and a stabilizer. Tablets and pills may be coated with an enteric or gastroenteric film, if necessary. They may be covered with two or more layers. They may also be adsorbed to a sustained release material, or microcapsulated. Additionally, the compositions may be capsulated by means of an easily degradable material such gelatin. They may be further dissolved in an appropriate solvent such as fatty acid or its mono, di or triglyceride to be a soft capsule. Sublingual tablet may be used in need of fast-acting property.

Examples of liquid compositions for oral administration include emulsions, solutions, suspensions, syrups and elixirs and the like. The composition may further contain a conventionally used inactive diluents e.g. purified water or ethyl alcohol. The composition may contain additives other than the inactive diluents such as adjuvant e.g. wetting agents and suspending agents, sweeteners, flavors, fragrance and preservatives.

The composition of the present invention may be in the form of spraying composition, which contains one or more active ingredients and may be prepared according to a known method.

Example of the intranasal preparations may be aqueous or oily solutions, suspensions or emulsions comprising one or more active ingredient. For the administration of an active ingredient by inhalation, the composition of the present invention may be in the form of suspension, solution or emulsion which can provide aerosol or in the form of powder suitable for dry powder inhalation. The composition for inhalational administration may further comprise a conventionally used propellant.

Examples of the injectable compositions of the present invention for parenteral administration include sterile aqueous or non-aqueous solutions, suspensions and emulsions. Diluents for the aqueous solution or suspension may include, for example, distilled water for injection, physiological saline and Ringer's solution.

Non-aqueous diluents for solution and suspension may include, for example, propylene glycol, polyethylene glycol, vegetable oils such as olive oil, alcohols such as ethanol and polysorbate. The composition may further comprise additives such as preservatives, wetting agents, emulsifying agents, dispersing agents and the like. They may be sterilized by filtration through, e.g. a bacteria-retaining filter, compounding with a sterilizer, or by means of gas or radioisotope irradiation sterilization. The injectable composition may also be provided as a sterilized powder composition to be dissolved in a sterilized solvent for injection before use.

The present external agent includes all the external preparations used in the fields of dermatology and otolaryngology, which includes ointment, cream, lotion and spray.

Another form of the present invention is suppository or pessary, which may be prepared by mixing active ingredients into a conventional base such as cacao butter that softens at body temperature, and nonionic surfactants having suitable softening temperatures may be used to improve absorbability.

According to the present invention, the fatty acid derivatives of the present invention are useful for treating a tumor with a decreased level of the 15-hydroxy-prostaglandin dehydrogenase (15-PGDH) in a mammalian subject with an effective amount of a fatty acid derivative. Thus, in some embodiments,

a fatty acid derivative disclosed herein for use in treating a tumor with a decreased level of the 15-hydroxy-prostaglandin dehydrogenase (15-PGDH);
a pharmaceutical composition for treating a tumor with a decreased level of the 15-hydroxy-prostaglandin dehydrogenase (15-PGDH) comprising an effective amount of a fatty acid derivative disclosed herein; and
a method for treating a tumor with a decreased level of the 15-hydroxy-prostaglandin dehydrogenase (15-PGDH) in a mammalian subject, which comprises administering to the subject in need thereof an effective amount of a fatty acid derivative disclosed herein,
are provided.

The term “treating” or “treatment” used herein includes prophylactic and therapeutic treatment, and any means of control such as prevention, care, relief of the condition, attenuation of the condition, arrest of progression, etc.

The term ‘tumor”, also known as a neoplasm used herein refers to any abnormal mass of cells or tissues which may be solid or fluid-filled. The tumor may be benign (not cancerous), potentially malignant, pre-malignant (pre-cancerous), or malignant (cancerous). The examples of the benign, potentially malignant or pre-malignant tumor used herein include, but not limited to, adenoma, polyp and teratoma. The examples of the cancerous tumor (cancer) used herein include, but not limited to, esophageal carcinoma, gastric carcinoma, duodenal cancer, small intestinal cancer, appendiceal cancer, large bowel cancer, colon cancer, rectum cancer, anal carcinoma, pancreatic cancer, liver cancer, gallbladder cancer, spleen cancer, renal carcinoma, bladder cancer, prostatic carcinoma, testicular carcinoma, uterine cancer, ovarian cancer, mammary carcinoma, pulmonary carcinoma and thyroid carcinoma and the metastasis thereof. Preferable example of cancer to be treated is colon cancer. According to the present invention, the fatty acid derivatives of the present invention are useful for suppressing the growth of tumor cell(s).

The term “decreased level(s) of the 15-hydroxy-prostaglandin dehydrogenase (15-PGDH)” means reduced, suppressed or down-regulated level(s) of 15-PGDH expression or activity compared to a healthy population. The decreased level(s) are for example, less than 95%, 90%, 85%, 80%, 75%, 70%, 65%, 60%, 55%, 50%, 45%, 40%, 35%, 30%, 25%, 20%, 15%, 10%, 5% or substantially 0% of the level(s) of 15-PGDH determined from a healthy population.

In another embodiment, the present invention relates to a method for suppressing the growth of a tumor cell with a decreased level of the 15-hydroxy-prostaglandin dehydrogenase (15-PGDH), which comprises contacting said tumor cell with an effective amount of a fatty acid derivative disclosed herein. In this embodiment, the method is conducted in vitro or in vivo. A fatty acid derivative disclosed herein for use in suppressing the growth of a tumor cell with a decreased level of the 15-hydroxy-prostaglandin dehydrogenase (15-PGDH); and a pharmaceutical composition for suppressing the growth of a tumor cell with a decreased level of the 15-hydroxy-prostaglandin dehydrogenase (15-PGDH) comprising a fatty acid derivative disclosed herein are also provided.

In further embodiment, the present invention relates to a method or a kit for identifying a subject who would be responsive to a fatty acid derivative disclosed herein, comprising,

(i) obtaining a biological sample from the subject; and
(ii) measuring 15-hydroxyprostaglandin dehydrogenase (15-PGDH) level. A step (iii) described below:
(iii) if the 15-PGDH level in the sample are decreased, administering to the subject an fatty acid derivative disclosed herein,
may be further added after the step (ii) of the above-described method to treat a tumor with a decreased level of the 15-PGDH. In this case, the method is considered to be a method for treating a tumor with a decreased level of the 15-PGDH after identifying a subject who is likely to be responsive to the fatty acid derivative.

The biological sample may be a tumor tissue. In some embodiments, the biological sample is a bodily fluid selected from blood, serum, plasma, a blood-derived fraction, stool, urine, and so on. In other embodiments, the blood-derived fraction comprises peripheral blood leukocytes. The measurement/detection of 15-PGDH level can be made by the conventional manner. For example, 15-PGDH level may be determined by Real-Time RT-PCR, Western Blot Analysis, ELISA, RIA or immunoprecipitation methood. The specific primers for the amplification of 15-PGDH and/or the specific antibody against 15-PGDH may be included in a kit for determining 15-PGDH level, which can be used for the method provided by the present invention. In the kit, reverse transcriptase (e.g. for Real-Time RT-PCR and/or nitrocellulose membrane (e.g. for Western Blot) may be further included. In one embodiment, when as a result of conducting the method provided by the present invention, 15-PGDH level Of the biological sample from the subject is found to be decreased compared to 15-PGDH level of a biological sample from a healthy volunteer, the subject is determined to be a subject who is likely to be responsive to the fatty acid derivative disclosed herein.

The pharmaceutical composition of the present invention may contain a single active ingredient or a combination of two or more active ingredients, as far as they are not contrary to the objects of the present invention.

In a combination of plural active ingredients, their respective contents may be suitably increased or decreased in consideration of their therapeutic effects and safety.

The term “combination” used herein means two or more active ingredient are administered to a patient simultaneously in the form of a single entity or dosage, or are both administered to a patient as separate entities either simultaneously or sequentially with no specific time limits, wherein such administration provides therapeutically effective levels of the two components in the body, preferably at the same time.

The present invention will be described in detail with reference to the following example, which, however, is not intended to limit the scope of the present invention.

### Example 1

Approximately half of colon cancers have a low level of NAD⁺-linked 15-hydroxyprostaglandin dehydrogenase (NAD⁺-15-PGDH). It was tested whether lubiprostone affected the growth of LS174T colon adenosarcoma cells, which are known to have a low level of 15-PGDH. LS174T cells (ATCC #CL-188) were grown in Eagle's Minimum Essential Medium (ATCC #30-2003) containing 10% Fetal Bovine Serum (ATCC #30-2020) at 37° C. Human PGDH (hPGDH) was expressed in LS174T cells using pCMV6-Entry Vector. Both the human cDNA clone (#RC204160) and the Precision shuttle vector (#PS100001) were from OriGene Technologies, Rockville, Md. LS174T cells were transfected with hPGDH cDNA using Lipofectamine and selected using G418 (200 μg/ml). LS174T cells±hPGDH (+hPGDH or + vector alone, mock) were grown in 35 mm petri dishes (0.5×104 cells/dish) in triplicate. Confluent non-transfected cells were plated in 6 well dishes (1×104 cells/well) in triplicate. Cultures were grown to 40% confluence and were then treated with either 0.1% EtOH-0.01% DMSO or 100 nM lubiprostone. Media were changed and fresh EtOH-DMSO or lubiprostone was added every 24 hr. Cell growth was monitored by directly counting the cells in a randomly chosen field using a cell cytometer at 0, 24, 48, 72 and 96 hr.

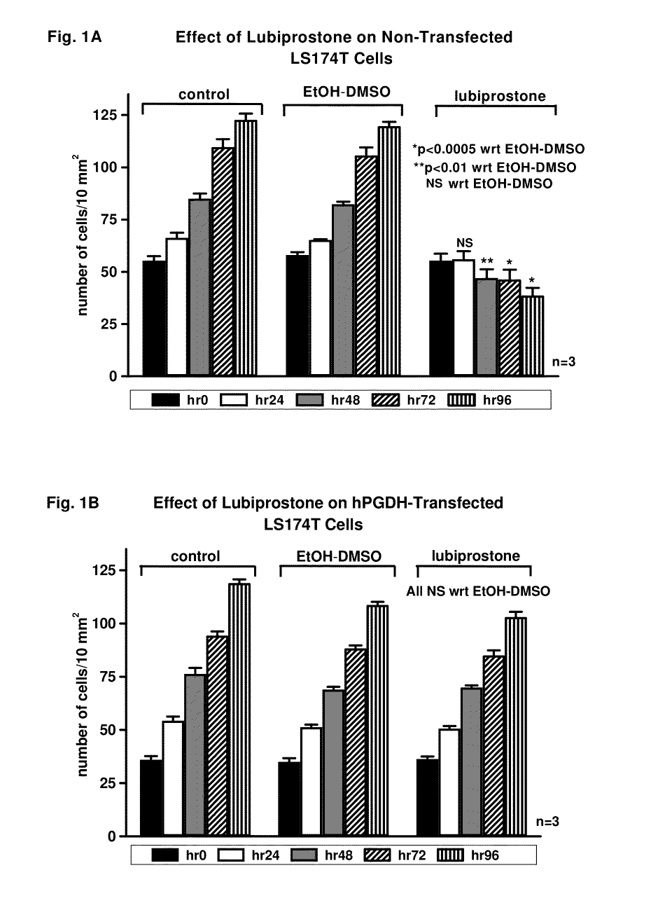

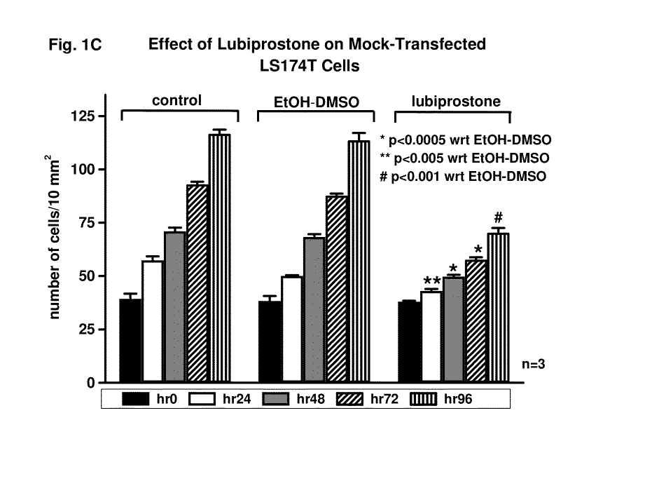

LS174 T cell growth was slowed or stopped by 100 nM lubiprostone (FIG. 1A). Transfection of LS174T cells with 15-OH PGDH prevented lubiprostone inhibition of cell growth (FIG. 1B). Mock transfection had no effect (FIG. 1C).

The data indicates that lubiprostone suppresses the growth of colon cancer cells with decreased levels of the 15-PGDH.

1. A method for treating a tumor with a decreased level of the 15-hydroxy-prostaglandin dehydrogenase (15-PGDH) in a mammalian subject, which comprises administering to the subject in need thereof an effective amount of a fatty acid derivative represented by the formula (I): 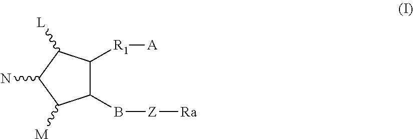
wherein L, M and N are hydrogen, hydroxy, halogen, lower alkyl, hydroxy(lower)alkyl, lower alkanoyloxy or oxo, wherein the five-membered ring may have at least one double bond;
A is —CH₃, or —CH₂OH, —COCH₂OH, —COOH or a functional derivative thereof;
B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;
Z is 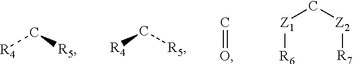
or single bond
wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;
R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

2. The method as described in claim 1, wherein Z is C═O.

3. The method as described in claim 1, wherein B is —CH₂—CH₂—.

4. The method as described in claim 1, wherein B is —CH₂—CH₂— and Z is C═O.

5. The method as described in claim 1, wherein L is hydroxy or oxo, M is hydrogen or hydroxy, N is hydrogen, B is —CH₂—CH₂— and Z is C═O.

6. The method as described in claim 1, wherein Ra is saturated C4-C7 (e.g. C5 or C6) aliphatic hydrocarbon residue substituted with one or more halogens (e.g. one or two halogens).

7. The method as described in claim 1, wherein R1 is a saturated bivalent straight or branched C5-C9 (e.g. C6 or C7) aliphatic hydrocarbon residue.

8. The method as described in claim 1, wherein the fatty acid derivative is (−)-7-[(2R,4aR,5R,7aR)-2-(1,1-difluoropentyl)-2-hydroxy-6-oxooctahydrocyclopenta[b]pyran-5-yl]heptanoic acid or (−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid, its isomers thereof or its functional derivative thereof.

9. The method as described in claim 1, wherein the fatty acid derivative is (−)-7-[(2R,4aR,5R,7aR)-2-(1,1-difluoropentyl)-2-hydroxy-6-oxooctahydrocyclopenta[b]pyran-5-yl]heptanoic acid.

10. The method as described in claim 1, wherein the tumor is colon cancer.

11. A method for identifying a subject who would be responsive to a fatty acid derivative represented by the formula (I) described in claim 1, comprising,
(i) obtaining a biological sample from the subject; and
(ii) measuring 15-hydroxyprostaglandin dehydrogenase (15-PGDH) level.

12. The method as described in any one of claims 1-9, wherein the pharmaceutical composition is administered to the subject identified according to the method as described in claim 11.

13. A method for suppressing the growth of a tumor cell with a decreased level of the 15-hydroxy-prostaglandin dehydrogenase (15-PGDH), which comprises contacting the tumor cell with an effective amount of a fatty acid derivative represented by the formula (I) described in claim 1.

14. The method as described in claim 13, wherein the method is conducted in vitro.

15. A kit for identifying a subject who would be responsive to a fatty acid derivative represented by the formula (I) described in claim 1 and treating the subject with the fatty acid derivative, comprising the fatty acid derivative represented by the formula (I) described in claim 1.
